(19) United States
(12) Patent Application Publication
(10) Pub. No.: US 2025/0271518 A1
(43) Pub. Date: Aug. 28, 2025

(54) SYSTEM AND METHOD FOR HYBRID MAGNET DESIGN FOR MAGNETIC RESONANCE IMAGING SYSTEMS

(71) Applicant: The General Hospital Corporation, Boston, MA (US)

(72) Inventors: Lawrence L. Wald, Charlsetown, MA (US); Clarissa Z. Cooley, Charlestown, MA (US); Alex C. Barksdale, Charlestown, MA (US)

(21) Appl. No.: 18/858,286

(22) PCT Filed: Apr. 21, 2023
(86) PCT No.: PCT/US2023/066058

§ 371 Date: Oct. 18, 2024

Related U.S. Application Data
(60) Provisional application No. 63/333,235, filed on Apr. 21, 2022.

(51) Int. Cl. G01R 33/34; G01R 33/00; G01R 33/3815; G01R 33/383; G01R 33/385

(52) U.S. Cl. CPC G01R 33/34023; G01R 33/0052; G01R 33/3403; G01R 33/3815; G01R 33/383; G01R 33/385

(57) ABSTRACT

Systems and methods are provided for a hybrid superconducting-permanent magnet MRI system. Permanent magnet elements may be used to supplement or shape the magnetic field produced by the superconducting windings. The hybrid design may increase field homogeneity or reduce the required bore length, thereby reducing the total system cost.

### CROSS-REFERENCE TO RELATED APPLICATION

The application is based on and claims priority to U.S. provisional application No. 63/333,235 filed on Apr. 21, 2022, which is hereby incorporated by reference herein in its entirety.

### GOVERNMENT-SPONSORED RESEARCH AND DEVELOPMENT

This invention was made with government support under 5U01EB025121-05 awarded by the National Institutes of Health. The government has certain rights in the invention.

### BACKGROUND

Typical superconducting magnet based magnetic resonance imaging (MRI) systems are designed with long and narrow-diameter cylindrical patient-bores. The confined space often leads to claustrophobia and increased patient motion, in-turn leading to costly increased scanning times, and recall rates. Bore length and diameter also limits the ability to perform other clinical procedures during imaging, such as patient monitoring, image-guided biopsy or other interventional procedures. To address this, manufacturers have constructed MRI systems with larger bore diameters to accommodate larger patients and seek the shortest bore length obtainable subject to other constraints, such as field uniformity over the imaging region. Magnet design is, therefore, a tradeoff between these parameters and constraints, as well as cost (largely determined by the wire length required to achieve the design).

Thus, there is a need for more flexible tradeoffs between magnet design parameters to produce cost effective imaging systems with shortened length while retaining other constraints.

### SUMMARY OF THE INVENTION

The present disclosure overcomes the aforementioned drawbacks by providing systems and methods for hybrid magnet design for magnetic resonance imaging (MRI) systems. In some non-limiting examples, non-superconducting magnets may be used to shape or supplement a static magnetic field produced by a superconducting magnet system. In one example, permanent magnets may be used to shape or supplement the static magnetic field during MRI imaging.

In one aspect, provided herein is a system for MRI that includes a bore, which can receive a subject for imaging with MRI. The system also includes superconducting wires that form a solenoid and produce a main magnetic field within the bore. A cryostat can be used to maintain the superconducting wires at a temperature that allows the superconducting wires to maintain a superconducting operational state for the MRI acquisition. The system further includes a plurality of permanent magnet elements that are configured to supplement or shape the main magnetic field. A gradient coil set may be included to apply a magnetic gradient. A radiofrequency system may also be included to transmit and receive radiofrequency signals during imaging.

In another aspect a system for MRI is provided that includes a bore, which can receive a subject for imaging with MRI. The system also includes superconducting wires that form a solenoid and produce a main magnetic field within the bore. A cryostat can be used to maintain the superconducting wires at a temperature that allows the superconducting wires to maintain a superconducting operational state for the MRI acquisition. The system further includes a plurality of permanent magnet elements that are configured to supplement or shape the main magnetic field.

Also provided herein is a method of manufacturing a hybrid MRI system. The method includes providing a bore and arranging a superconducting magnet system and one or more rings of permanent magnet elements. The superconducting magnet system may be placed proximate to the bore to produce a static magnetic field within the bore. The permanent magnet elements may be placed proximate to the bore in order to supplement or shape the static magnetic field.

In other aspects, a method for manufacturing a hybrid MRI system is provided. The method includes constructing a bore that has a center axis and arranging rings of magnet elements around the bore. The rings may be arranged at a first radial distance between the center axis and the rings. The method also includes arranging superconducting windings around the bore at a second radial distance that describes the distance between the center axis and the windings. Further, the method includes manipulating the positions along the center axis of the rings, the superconducting windings, or both to control a required total current in the superconducting windings or an achieved magnetic field profile within a target imaging volume.

### DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 is a block diagram of an example hybrid MRI system that can utilize the systems or implement the methods described in the present disclosure.

FIG. 2A is an illustration of the components of a standard superconducting magnet design for a magnetic resonance imaging (MRI) system.

FIG. 2B is an illustration of a hybrid magnet design in accordance with the present disclosure.

FIG. 3A is an illustration of permanent magnets arranged in an Aubert ring pair according to some aspects of the present disclosure.

FIG. 3B shows an example of the magnetic field produced by the ring pair configuration of FIG. 3A according to simulation.

FIG. 3C is an illustration of permanent magnets arranged in a parallel ring pair according to some aspects of the present disclosure.

FIG. 3D shows an example of the magnetic field produced by the ring pair configuration of FIG. 3C according to simulation.

FIG. 4 illustrates the generalized orientation of each permanent magnet element with respect to the z axis.

FIG. 5A illustrates a discretized spatial model of the hybrid magnet design used for optimization in accordance with some aspects of the disclosure.

FIG. 5B is a block diagram illustrating the design process in accordance with some aspects of the disclosure.

FIG. 6A shows an example of simulated B₀ homogeneity contours produced by a standard superconducting magnet system.

FIG. 6B shows an example of simulated B₀ homogeneity contours produced by an Aubert ring pair of permanent magnets.

FIG. 6C shows an example of simulated B₀ homogeneity contours produced by a hybrid magnet system that combines superconducting windings with Aubert ring pairs.

FIG. 7A illustrates tradeoff curves for an example Aubert configuration (θ=90°) over increasing allowed permanent magnet material mass.

FIG. 7B illustrates tradeoff curves for 100 kg permanent magnet material constraints for different angle configurations.

FIG. 7C illustrates tradeoff curves for 250 kg, permanent magnet material constraints for different angle configurations.

FIG. 7D illustrates tradeoff curves for 500 kg permanent magnet material constraints for different angle configurations.

FIG. 8 is a block diagram of an example system for generating images with a hybrid MRI system and designing a hybrid MRI system according to some aspects described in the present disclosure.

FIG. 9 is a block diagram of example components that can implement the system of FIG. 8.

### DETAILED DESCRIPTION

Before any aspects of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Magnetic resonance imaging (MRI) systems typically use superconducting magnets in which solenoid coils are cooled down to very low temperatures to achieve minimal resistance and maximum field strength. These magnets require cooling systems to maintain an extremely low temperature, such as around 4 K. The cryogen systems required to maintain these systems are complex and extremely expensive, particularly, if quenched. Moreover, to achieve maximal B₀ field homogeneity in the scanning region, the bore sizes are typically long and have small diameters, both parameters contribute to claustrophobia or challenges with fitting larger patients in the bore.

In addition to improving patient compliance and clinical utility, there is a need for cheaper MRI systems that have lower power and siting requirements to expand access to MRI. While most clinical scanners have field strengths of at least 1.5 T, mid-field (e.g., 0.3 T to 1.0 T) and low-field (<0.3 T) systems are emerging as lower-cost alternatives. Reducing the bore length can also reduce costs with the rising price of superconducting wire. Thus, there is a clinical need for short, wide-bore magnets at lower field strengths for more flexible and less expensive MRI exams. The present disclosure provides a flexible design that combines superconducting magnets with other, non-superconducting magnet elements. This design can reduce overall costs and allow for shorter bore lengths, while yielding the same or similar clinical utility. The design may also be used to improve the homogeneity of the main magnetic field (B₀).

MRI scanners based on mid-field (e.g., 0.3 T to 1.0 T) superconducting solenoid magnets have emerged as an attractive compact scanning option with the potential to increase accessibility where system and operating costs or siting footprint presents a barrier. Furthermore, their lower field strength offers the potential for increasing the range of implants that can be scanned due to either safety or susceptibility artifact improvements. Finally, the >3-times lower field strengths can improve patient comfort and acceptance by allowing shorter bore or larger diameter magnets.

The trade-off between magnet length and superconducting wire length, which can be considered a proxy for cost, has been examined in optimal solenoid designs for a given bore diameter, imaging diameter of spherical volume (DSV), and homogeneity target. The disclosed system and method can be used to further reduce the bore length for a superconducting magnet design by employing non-superconducting magnets inside the bore to supplement and shape the field from the superconducting windings. This hybrid design can be used to preserve the performance metrics of superconducting magnets by jointly optimizing the positioning of the other (non-superconducting) magnet rings and the superconducting winding placement. As will be described, in one non-limiting example, the non-superconducting magnets may include permanent magnets, which may be rare-earth permanent magnets. Permanent magnets may be advantageous because they do not require, for example, electrical or electromechanical or electromagnetic systems to shape or supplement the field created by the superconducting magnet. However, other non-superconducting magnets or non-superconducting magnet elements may be used, for example, such as electromagnets or the like.

The use of permanent magnets along with a superconducting solenoid can be used for mid- (e.g., 0.3 T to 1.0 T) to low- (<0.3 T) field MRI systems without compromises of clinical performance because high remanence and coercivity materials allow for significant field contributions relative to the superconducting windings, while retaining their original magnetization strength and direction. As will be described, the placement of permanent magnet materials and superconducting windings can be jointly optimized to achieve a target field.

In some configurations, various types of ring arrangements may be used for the permanent magnet elements, which can be, for example, placed inside the superconducting solenoid. In some configurations an Aubert ring pair can be used, in which the magnetic moments of the permanent magnet elements can be oriented radially. In some configurations, a ring pair with axial magnetization parallel or antiparallel to B₀ can be utilized. In some configurations, the magnet elements may have arbitrary orientations that can be described by an angle, θ. Examples are provided, comparing the performance of the hybrid magnet design (i.e., superconducting combined with permanent magnets) with a typical superconducting magnet design.

For exemplary and explanatory purposes only, the systems and methods described herein utilize the non-limiting example of a 0.5 T target field. However, it should be understood that the methods and systems described herein are not limited to such a field strength. Rather, as will be described, the systems and methods provided herein readily apply to a wide variety of field strengths and systems, including other low- and mid-field strengths. Any field strength that does not re-orient the magnetic moment of the permanent magnet materials may utilize the systems and methods described herein.

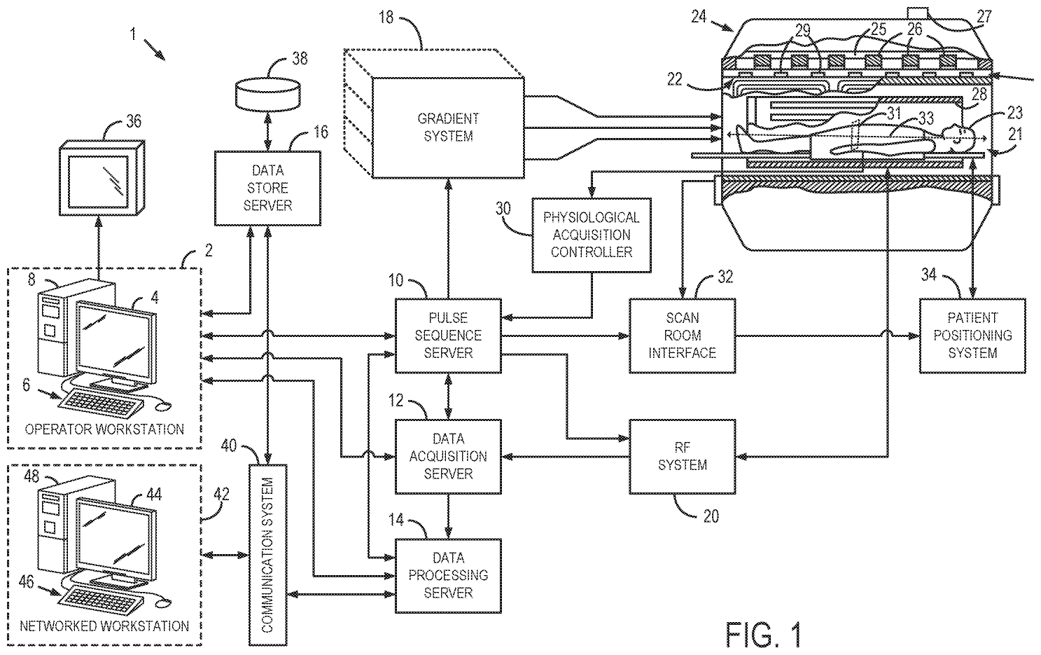

Referring particularly now to FIG. 1, an example of a hybrid MRI system 1 that can implement the methods described here is illustrated. The hybrid MRI system 1 can include a superconducting magnet system and a non-superconducting magnet system, which may include permanent magnet elements. The MRI system may include a bore 21, which may receive a subject 23, which may be a patient, phantom, or other subject during scanning.

The MRI system 1 may have an operator workstation 2 that may include a display 4, one or more input devices 6 (e.g., a keyboard, a mouse), and a processor 8. The processor 8 may include a commercially available programmable machine running a commercially available operating system. The operator workstation 2 provides an operator interface that facilitates entering scan parameters into the MRI system 1. The operator workstation 2 may be coupled to different servers, including, for example, a pulse sequence server 10, a data acquisition server 12, a data processing server 14, and a data store server 16. The operator workstation 2 and the servers 10, 12, 14, and 16 may be connected via a communication system 40, which may include wired or wireless network connections.

The pulse sequence server 10 functions in response to instructions provided by the operator workstation 2 to operate a gradient system 18 and a radiofrequency (“RF”) system 20. Gradient waveforms for performing a prescribed scan are produced and applied to the gradient system 18, which then excites gradient coils in an assembly 22 to produce the magnetic field gradients (e.g., Gₓ, G_{y}, and G_{z}) in addition to the main magnetic field. The gradients may be used for spatially encoding magnetic resonance signals or for producing desired contrast. The gradient coil assembly 22 forms part of a magnet assembly 24 that includes a polarizing magnet 26 and a whole-body RF coil 28.

The hybrid MRI system 1 may include a superconducting magnet system 26, which can be configured to produce a main magnetic field within the bore 21. For example, the superconducting magnet system 26 may be configured to produce a static main magnetic field of B₀=0.5 T that is maximally homogeneous within the bore 21 or a subspace of the bore 21, such as a 40 cm sphere at the center of the bore. The superconducting magnet system 26 may include a superconducting wire or several superconducting wires wound around the bore forming a solenoid. The superconducting wire may contain copper, niobium-titanium (NbT), or other suitable materials. The hybrid MRI system may also include a cryostat 25. The cryostat 25 may keep the superconducting magnet system 26 at a temperature that maintains the superconducting wires in a superconducting operational state. For example, the cryostat 25 may be filled with liquid helium to maintain the superconducting windings at a temperature <10 K, such as ˜4 K. The hybrid MRI system 1 may also include a cold head 27, which may provide access to the cryostat 25 for filling with liquid helium, provide access for heat exchange, work with a compressor system to recycle helium, or otherwise function to maintain a low temperature of the cryostat 25 and superconducting magnet system 26.

The hybrid MRI system 1 may further include non-superconducting magnets or permanent magnet elements 29 that can be used to supplement or shape the main magnetic field produced by the superconducting magnet system 26. The permanent magnet elements 29 may be arranged outside of and/or around the bore 21. For example, the permanent magnet elements 29 may be placed near or proximate to the bore 21 and may be placed between the wall of the bore 21 and the superconducting magnet system 26. In one non-limiting example, the permanent magnet elements 29 may be arranged outside of the cryostat 25. In this way, the permanent magnet elements 29 may not be cooled or be less cooled. In one non-limiting example, insulation 35 may be provided between the cryostat 25 and the permanent magnet elements 29. The insulation 35 may be air, or may be an insulating material. In other configurations, the permanent magnet elements 29 may be cooled or subject to the cold of the cryostat 25.

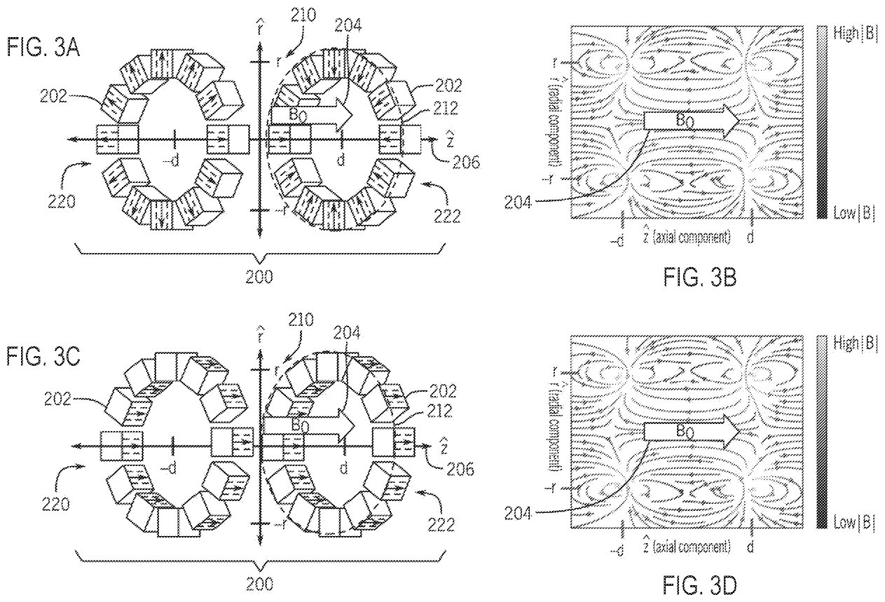

As will be described in further detail below with respect to FIGS. 3A and 3C, the permanent magnet elements 29 may be arranged in various ways. As a non-limiting example, the permanent magnet elements 29 may be arranged in one or more rings or partial rings. The permanent magnet elements 29 may be evenly or otherwise spaced around the circumference 212 of the ring. The rings may be placed near or proximate to the bore 21 such that the circumference 212 of each ring extends around the circumference of the bore 21, fully or partially surrounding the circumference of the bore 21. The rings may be circular, nearly circular, or shaped otherwise. The rings may be placed in pairs around the circumference of the bore 21 providing symmetry over the X-Y plane 31 along the center axis 33 (i.e., z-axis) of the bore 21 of the MRI system 1. For example, one ring may be placed at a position z=−d, and the other ring of the ring pair may be placed at a position z=+d.

The RF system 20 may apply RF waveforms to the RF coil 28, or a separate local coil, to perform the prescribed magnetic resonance pulse sequence. Responsive magnetic resonance signals detected by the RF coil 28, or a separate local coil, are received by the RF system 20. The responsive magnetic resonance signals may be amplified, demodulated, filtered, and digitized under direction of commands produced by the pulse sequence server 10. The RF system 20 includes an RF transmitter for producing a wide variety of RF pulses used in MRI pulse sequences to excite the magnetic resonance signal. The RF transmitter is responsive to the prescribed scan and direction from the pulse sequence server 10 to produce RF pulses of the desired frequency, phase, and pulse amplitude waveform. The generated RF pulses may be applied to the whole-body RF coil 28 or to one or more local coils or coil arrays.

The RF system 20 also includes one or more RF receiver channels. An RF receiver channel includes an RF preamplifier that amplifies the magnetic resonance signal received by the coil 28 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 10 may receive patient data from a physiological acquisition controller 30. By way of example, the physiological acquisition controller 30 may receive signals from a number of different sensors connected to the patient, including electrocardiograph (“ECG”) signals from electrodes, or respiratory signals from a respiratory bellows or other respiratory monitoring devices. These signals may be used by the pulse sequence server 10 to synchronize, or “gate,” the performance of the scan with the subject's heart beat or respiration.

The pulse sequence server 10 may also connect to a scan room interface circuit 32 that receives signals from various sensors associated with the condition of the patient and the magnet system. Through the scan room interface circuit 32, a patient positioning system 34 can receive commands to move the patient to desired positions during the scan.

The digitized magnetic resonance signal samples produced by the RF system 20 are received by the data acquisition server 12. The data acquisition server 12 operates in response to instructions downloaded from the operator workstation 2 to receive the real-time magnetic resonance data and provide buffer storage, so that data is not lost by data overrun. In some scans, the data acquisition server 12 passes the acquired magnetic resonance data to the data processor server 14. In scans that require information derived from acquired magnetic resonance data to control the further performance of the scan, the data acquisition server 12 may be programmed to produce such information and convey it to the pulse sequence server 10. For example, during pre-scans, magnetic resonance data may be acquired and used to calibrate the pulse sequence performed by the pulse sequence server 10. As another example, navigator signals may be acquired and used to adjust the operating parameters of the RF system 20 or the gradient system 18, or to control the view order in which k-space is sampled. In still another example, the data acquisition server 12 may also process magnetic resonance signals used to detect the arrival of a contrast agent in a magnetic resonance angiography (“MRA”) scan. For example, the data acquisition server 12 may acquire magnetic resonance data and processes it in real-time to produce information that is used to control the scan.

The data processing server 14 receives magnetic resonance data from the data acquisition server 12 and processes the magnetic resonance data in accordance with instructions provided by the operator workstation 02. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 14 are conveyed back to the operator workstation 02 for storage. Real-time images may be stored in a data base memory cache, from which they may be output to operator workstation 02 or a display 36. Batch mode images or selected real time images may be stored in a host database on disc storage 38. When such images have been reconstructed and transferred to storage, the data processing server 14 may notify the data store server 16 on the operator workstation 2. The operator workstation 2 may be used by an operator to archive the images, produce films, or send the images via a network to other facilities.

The MRI system 1 may also include one or more networked workstations 42. For example, a networked workstation 42 may include a display 44, one or more input devices 46 (e.g., a keyboard, a mouse), and a processor 48. The networked workstation 42 may be located within the same facility as the operator workstation 2, or in a different facility, such as a different healthcare institution or clinic.

The networked workstation 42 may gain remote access to the data processing server 14 or data store server 16 via the communication system 40. Accordingly, multiple networked workstations 42 may have access to the data processing server 14 and the data store server 16. In this manner, magnetic resonance data, reconstructed images, or other data may be exchanged between the data processing server 14 or the data store server 16 and the networked workstations 42, such that the data or images may be remotely processed by a networked workstation 42.

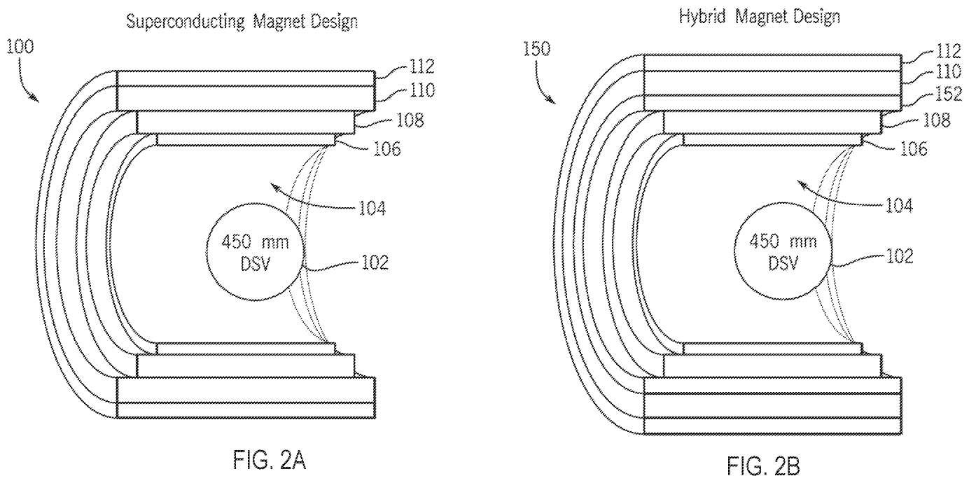

Referring now to FIGS. 2A and 2B, example configurations of the magnet assembly 24 are shown in further detail. FIG. 2A shows a standard superconducting magnet design 100. FIG. 2B shows an illustration of a hybrid magnet design 150, which can be compared to the standard superconducting magnet design 100 FIG. 2A. Both the superconducting magnet design 100 and hybrid magnet design 150 may include a target imaging volume or DSV 102, a magnet or patient bore 104, a body tube 106, a gradient coil set 108, a cryostat layer 110, and a superconducting winding space 112. The superconducting winding space 112 may be filled with a superconducting magnet system, as will be described in detail below.

The hybrid magnet design 150 may further include a layer of magnet space 152, which can be filled with permanent magnet elements, as will be described in detail below. The magnet space 152 may accommodate a rare-earth magnet and, thus, may be called a rare-earth magnet space 152, too. However, non-rare-earth magnets may be used. The magnet space 152 may be situated between the gradient coil set 108 and the superconducting winding space 112. In a non-limiting example, the magnet space 152 may be arranged outside of the bore 104, the cryostat layer 110 may be arranged outside of the magnet space 152, and the superconducting winding space 112 may be arranged in the cryostat layer 110. However, in some configurations, the relative position of the gradient coil set 108, superconducting winding space 112, or magnet space 152 may be modified.

The target imaging DSV 102 may be used to describe a scanning region within the patient bore 104 in which the field can be optimized to be homogeneous. The target imaging DSV 102 may be defined specifically based on the target anatomy of the system or be defined as a larger region for broader applications. For example, for head or knee imaging, the targeting imaging DSV 102 may be a 450 mm sphere at the center of the patient bore 104, as shown. Alternatively, for imaging larger volumes, such as abdomen imaging, the target imaging DSV may be defined as a subspace of the patient bore 104 of any desired shape.

The body tube 106 may include a body radiofrequency (RF) coil and an RF shield, such as illustrated in FIG. 1. The RF coil may be a body transmit RF coil, a receive RF coil, or a transmit/receive RF coil. The body RF coil may also be omitted if localized RF coils, such as head coils, knee coils, and the like, are used. The body RF coil may also be used as the transmit RF coil, while localized coils can be used as the receive coils.

The gradient coil set 108 can be used to produce magnetic field gradients along the x, y, z, or oblique axes. In the hybrid magnet design 150, the gradient coil set 108 can be located on the inside of the magnet space 152, as shown in FIG. 2B. The gradient coil set 108 can alternatively be located on the outside of the magnet space 152.

The superconducting winding space 112 may contain superconducting wire that winds to form a solenoid winding and produce a main magnetic field (B₀). The cryostat layer 110 may be filled with liquid helium or another cooling agent to keep the superconducting winding at a cold temperature suitable for superconducting. It may be located on the inside of the superconducting winding space 112, as shown in FIG. 2B, on the outside of the superconducting winding space 112, or on both sides of the superconducting winding space 112. Other layers, such as vacuum space, insulators, and the like, may be present in conjunction with the cryostat layer 110 to keep the superconducting winding space 112 at a sufficiently cold temperature to maintain superconducting properties.

As a nonlimiting example, the layers within the superconducting magnet design 100 and hybrid magnet design 150 may have the following widths:

| | Superconducting | Hybrid magnet design |
|---|---|---|
| Component | magnet design 100 | 150 |
| Target imaging DSV 102 | 450 mm sphere | 450 mm sphere |
| Patient bore 104 | 700 mm | 700 mm |
| Body tube 106 | 780 mm | 780 mm |
| Gradient coil set 108 | 940 mm | 940 mm |
| Magnet space 152 | — | 1060 mm |
| Cryostat layer 110 | 1120 mm | 1240 mm |
| Superconducting winding space 112 | 1220 mm | 1340 mm |

The magnet space 152 may be filled with rare-earth or other permanent magnet elements in a variety of arrangements and orientations to supplement or shape the main magnetic field produced by the superconducting winding. In a non-limiting example, the permanent magnet elements may be constructed from a magnetic material like neodymium (NdFeB or Nd₂Fe₁₄B), samarium-cobalt (SmCo or Sm(Co,Fe,CuZr)₇), or the like. The magnet elements may be of various sizes and shapes and arranged into ring pair formations that can be positioned within the magnet space 152. For example, the magnet elements may include 2-cm cubical blocks arranged into 50 rings with 48 permanent magnet elements in each ring. The rings may be placed within the magnet space 152. The magnet elements may also have another non-cubical prism shape or any other desired shape. In general, 1 to 100 or more rings can be positioned within ring pairs placed in the magnet space 152 depending on the size of the magnet elements and the bore length. The rings may be placed at various locations along the bore. In one example implementation, the magnet elements may be arranged symmetrically along the bore (i.e., symmetric along the z-axis across the x-y plane). In other configurations, the arrangements may be asymmetrical. Each magnet element may have the same orientation within the ring, such as radial or parallel with respect to the central axis of the magnet bore. Alternatively, the orientation of each magnet element may be varied within the ring, by an arbitrary or predefined angle, θ, which will be described in more detail below. The orientation of the magnet elements may be chosen in order to achieve a desired magnetization.

Referring now to FIGS. 3A, 3B, 3C, and 3D, non-limiting examples of arrangements for magnet elements 202 within ring pairs 200 and their corresponding field profiles are shown. One or more of these ring pairs 200 may be placed into the magnet space 152 (FIG. 2B), and the positions of the rings 220 and 222 may be determined from a design process, which may include an optimization, as will be described in detail below. It should be understood that the disclosed orientations are supplied as examples and that, in practice, the orientation of the magnet elements may be positioned to maximally achieve any desired magnetic field. For example, the magnet elements may be arranged to produce magnetization in the radial direction, angled with respect to radial, parallel to the patient bore, anti-parallel to the patient bore, or the like.

The magnetic moment of each permanent magnet element 202 is represented by an arrow. The B₀ field 204 direction is labeled, which indicates the direction of the main magnetic field produced by the superconducting windings in the hybrid magnet design 150 once the ring pair 200 is placed into the magnet space 152. However, in some configurations the B₀ field may be oriented in another direction. The non-limiting examples of parallel, anti-parallel, and Aubert ring pairs 200 are shown. However, other arrangements can be used as well.

As shown in the examples (i.e., FIGS. 3A and 3C), two rings 220 and 222 may be situated at a distance 2d apart along a longitudinal axis 206 of the bore (e.g., z-axis). For example, one ring 220 may be situated at z=−d and one ring 222 may be situated at z=d to form a ring pair 200. The rings may include any number of permanent magnet elements 202, which may be constrained by the total weight and size of the permanent magnet elements 202. The permanent magnet elements 202 may be arranged in a ring 220 or 222 that surrounds or partially surrounds the bore. For example, the elements may be equally or otherwise spaced around the circumference 212 of the ring 220 or 222. The ring 220 or 222 may have a distance 210, r, that describes the distance from the z axis 206 to the permanent magnet elements 202. The ring 220 or 222 may be circular, in which r may describe the radius of the ring 220 or 222, or nearly circular, such that the radial distance 210 of the ring 220 or 222 is within ±20% of r. The ring 220 or 222 may alternatively be elliptical or another shape, in which r may describe the average radial distance 210 from the center of the ring 220 or 222 to the ring elements 202.

As a non-limiting example, the permanent magnet elements 202 can be equally spaced around the circumference 212 of the ring 220 or 222 in an Aubert ring pair configuration, as shown in FIG. 3A, which produces the corresponding field profile illustrated in FIG. 3B, according to simulation. The Aubert ring pair configuration shown in FIG. 3A is characterized by radially oriented magnetic moments. In one ring 220, the magnetic moments are outwardly oriented (i.e., toward r>0), and in the other ring 222, the magnetic moments are inwardly oriented (i.e., toward r=0).

As another non-limiting example, the permanent magnet elements 202 can be equally spaced around a ring 220 or 222, orienting the magnetic moments parallel to an axis parallel to the longitudinal axis 206 of the bore, which may also be the direction of the B₀ field 204, reminiscent of ferroshims. The field produced by the parallel arrangement of FIG. 3C, according to simulation, is shown in FIG. 3D. The parallel configuration shown in FIG. 3C is characterized by aligning the magnetic moments of both rings 220 and 222 along the z direction, parallel to the B₀ field produced by the superconducting magnet. Unlike ferroshims, the magnet elements 202 can also be oriented to flip the magnetic moments. Thus, the magnetic moments of both rings 220 and 222 may also be aligned antiparallel to an axis parallel to the longitudinal axis 206 of the bore.

A radially symmetric magnetic field is generated, as shown in FIGS. 3B and 3D, where the direction is indicated by the arrows and the magnitude is indicated by the grayscale bar. In the example arrangements, the generated magnetic field is along the z-direction 206, parallel or antiparallel to B₀ 204 of the superconducting magnet, and has the highest homogeneity centered between the ring pair 200 (i.e., z=0) and in the center of the ring 220 and 222 (i.e., r=0).

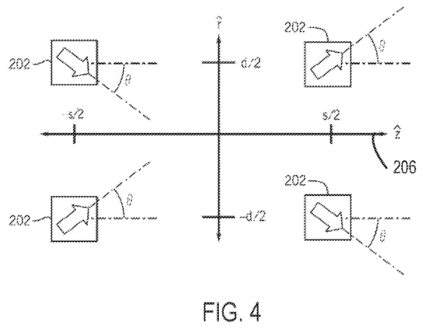

In general, each magnet element may be oriented within the ring 220 or 222 with an angle, θ, which may describe the angle of the magnetic moment with respect to the z axis 206, as shown in FIG. 4, in which the magnetic moment of each permanent magnet element 202 is represented by an arrow. For example, in an Aubert ring pair, as shown in FIG. 3A, θ=270°. As another non-limiting example, in a parallel configuration, as shown in FIG. 3C, θ=0°. For each ring, all of the magnet elements within the ring may have an equal orientation angle. Alternatively, the magnet elements may have varied orientation angles within the same ring. The orientation angles of the permanent magnet elements may be arbitrarily chosen (i.e., 0°<θ<360°) or may be a predefined set, such as [0°, 180°, 270°], or another predefined set.

For a given ring pair orientation (e.g., FIG. 3A, 3C, or the like), a design or manufacturing process can be used to determine the placement of multiple ring pairs 200 within the magnet space 152. In one configuration, this process may be an optimization. The process can further be used to control and select the orientation of the magnet elements 202 within the ring pair 200 by determining a desired (or, if preferred, an optimal) angle θ, as will be described.

In general, the design process can be used to arrange the superconducting magnet system and permanent magnet elements to control or minimize the total current required to achieve a desired magnetic field. Reducing the required current allows for reduced superconducting windings, and thereby greatly reduces the cost of the MRI system. Several magnet lengths can be assessed to characterize the tradeoffs between the required current, which can act as a surrogate for cost, and other parameters, such as superconducting winding diameter, bore length, and total weight of the permanent magnet elements 202 for a desired field target.

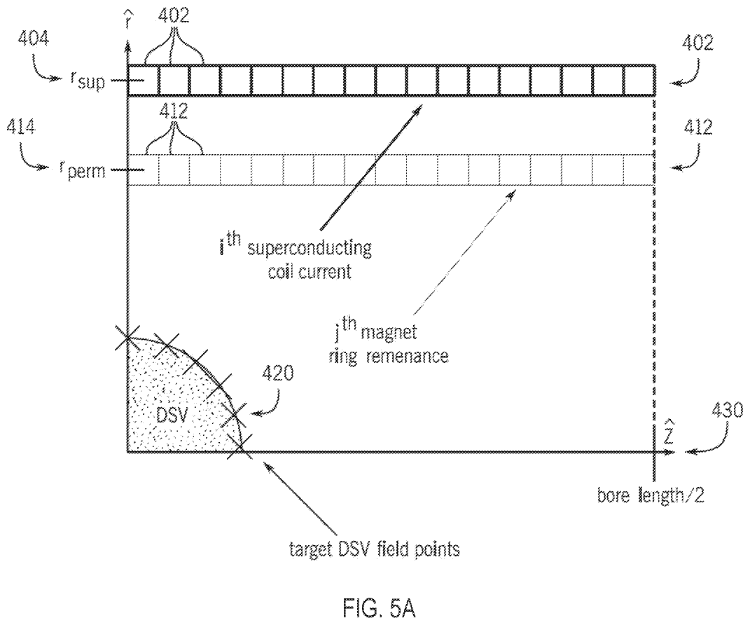

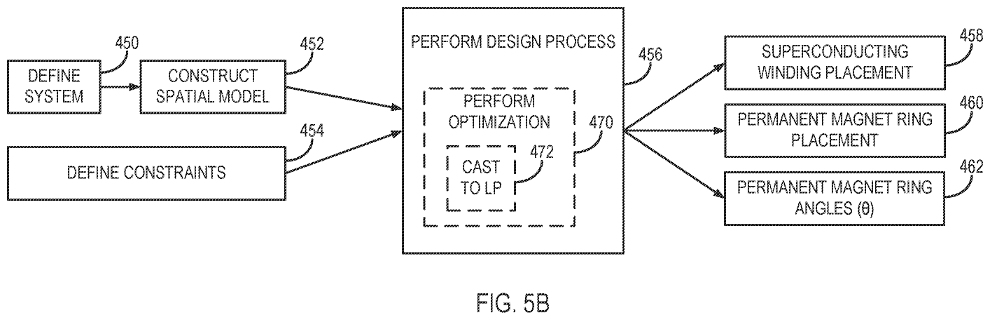

The design process can first be described in the context of the standard superconducting magnet design (FIG. 2A) and extended to include the permanent magnet elements 202 placed into the magnet space 152. The design process can be formulated as a linear programming problem, after discretizing a spatial model of the magnet system, as illustrated in FIG. 5A. For example, the system can be modeled in radial space, r, along the length of the bore, z 430. The potential locations for superconducting coil currents 402 can be discretized to include a given number (n) of discrete placeholders for superconducting coil currents 402 at a radial distance rₛᵤₚ 404 between the center of the bore or z-axis 430 and the superconducting magnet. The DSV may also be discretized to define discrete target DSV field points 420 corresponding to the target imaging volume DSV. For example, the target DSV field points can be defined as 41 points along a quarter circle arc of a central slice of the DSV, utilizing symmetry and properties of solutions to Laplace's equation in source-free regions. More points may be used to achieve a higher spatial resolution, or less points may be used to reduce the computational requirements.

The design process may then be used to populate currents into the placeholder locations for superconducting coil currents 402 to control the total current in the superconducting magnet system, the magnetic field profile in the imaging volume, or both. For example, the placement of superconducting coil currents 402 may achieve a target field along a set of target DSV field points 420 that line the perimeter of the DSV while minimizing the total power of the current loops. In general, minimizing the total volume of superconducting material is equivalent to minimizing total current, under the assumption of uniform current density of the superconducting windings.

The design process, which may be solved efficiently using standard linear programming packages, may be formulated as:

$${min}_{{x}_{1} , … , {x}_{n}} {\sum }_{i} \frac{2 π {r}_{sup}}{{J}_{curr}} \vert {x}_{i} \vert ⇒ {min}_{{x}_{1} , … , {x}_{n}} {\sum }_{i} \vert {x}_{i} \vert$$

subject to

$${B}_{z m i {n}^{′}} \leq {A}_{sup e r c o n} x \leq {B}_{z m ax}$$

where xᵢ is the current at the i^{th} candidate superconducting coil current 402, J_{curr} is the superconducting critical current density x is the vector of candidate currents, and |⋅| is the [symbol] ₁-norm. It can be assumed that all of the superconducting loops have equal diameters (dₛᵤₚ) and that superconducting loops are symmetric about z=0. The minimization may use other measures of the total superconducting current, such as the [symbol] ₂-norm, etc. A_{supercon} is the field at each DSV point per unit current from superconducting loop source, such that the (m, n) element is the field at the m^{th} DSV location from the n^{th} superconducting source location. B_{z min} and B_{z max} represent magnetic field constraints. For example, the magnetic field constrains may be based on a target field strength B₀ and homogeneity specification of ∈, where B_{z min}=B₀(1−0.5∈) and B_{z max}=B₀(1+0.5∈) in parts per million (ppm).

For axisymmetric designs, the radial component may be neglected when constraining the field, as the radial component is typically much smaller than the axial component. Thus, it may be sufficient to only impose constraints on B_{z}. Because the DSV is free of magnetic sources, B_{z} satisfies Laplace's equation, thus constraining only the surface of the DSV constrains the volume inside of the DSV as well. Further taking advantage of the axis-symmetry and symmetry about z=0, the entire DSV can be constrained by specifying field targets along a quarter arc of a circle on a cross-section in the \hat{r}-\hat{z} plane, dramatically reducing the number of DSV computation points used in the process.

This formulation can present advantages a over previous magnet design formulations. For example, the [symbol] ₁-norm of the superconducting loop currents promotes sparsity in the solution, naturally aiding in “buildability” of the desired magnet by producing a minimum number coil magnet and, if desired, optimality of solution given existence.

The design process can be extended to include permanent magnet elements in a ring pair. In this way the design process may be used to arrange permanent magnet elements to control the total current in the superconducting magnet system, the magnetic field profile in a target imaging volume, or both. For example, the placement of the permanent magnet elements may shape the field to provide a maximally homogeneous static magnetic field while minimizing the total current of the superconducting magnet system.

The ring pair may be parameterized by the magnetization angle, θ, as discussed in the context of FIG. 4. Two example configurations are presented in FIGS. 3A and 3C, including the Aubert ring configuration (e.g., FIG. 3A) with θ=270° and the parallel configuration (e.g., FIG. 3C) with θ=0°. Using permanent magnet elements, such as rare-earth materials, with sufficient coercivity enables arbitrary θ while retaining magnetization in the main B₀ field from the superconducting windings.

The model in FIG. 5A can be extended to include discretized candidate locations for permanent magnet elements placed in rings at discrete locations j with a radial distance rₚₑᵣₘ 414 between the center of the bore or z-axis 430 and the permanent magnet elements. This provides discrete candidate locations for permanent magnet ring remanence 412. In this way, the process can determine whether or not to populate each candidate location for permanent magnet ring remanence 412 with a permanent magnet ring.

The objective function can be extended to:

$${min}_{{x}_{1} , … , {x}_{n}} {\sum }_{i} \vert {x}_{i} \vert$$

subject to:

$${B}_{z m i n} \leq {A}_{sup e r c o n} x + {C}_{perm}^{θ} {y}^{θ} \leq {B}_{z m ax}$$

$${\sum }_{j} \vert {y}_{j}^{θ} \vert \leq {M}_{perm} / {m}_{perm}$$

The first constraint can ensure that the resulting magnetic field remains within a target magnetization profile, which may be described as a homogeneity specification (∈) of the target field strength (B₀). Here, yⱼ^{θ} is the permanent magnet decision variable for the j^{th} ring remanence candidate location, corresponding to permanent magnet rings with magnetization angle, θ, and y^{θ} is the vector containing all permanent magnet decision variables. Cₚₑᵣₘ^{θ} is the field produced per unit of permanent magnet material at the DSV points, such that the (m, n) element is the field at the m^{th} DSV location from the n^{th} permanent magnet source location.

The second constraint can be used to limit the overall mass of the permanent magnet elements. Here, Mₚₑᵣₘ is the total mass constraint set based on the practicality of building the rings and mₚₑᵣₘ is the discretized unit mass of the rings. Similarly, the second constraint can be used to limit the total number of permanent magnet elements.

The permanent magnet decision variable, yⱼ^{θ}, can scale the volume of the permanent magnet elements, the field strength of each permanent magnet element, Bₚₑᵣₘ, or a combination thereof. The volume of permanent magnets can also be scaled by scaling the 2 dimension of the rare-earth optimization units.

To solve, the magnetic field produced by superconducting coil currents 402 and permanent magnet ring remanences 412 can be calculated using any of a variety of tools, such as Magpylib, another suitable software package, or empirical measurement. If desired, optimization can be performed using linear programming methodology. For example, the [symbol] ₁-norm of superconducting currents and remanences can be cast into linear programming form by introduction of auxiliary variables, casting absolute values into two linear inequalities. The MATLAB (Natick, MA) linear programming toolbox (linprog), or other linear programming solvers, may be used to compute the optimized solutions over currents and remanences by superimposing the fields from candidate source locations. Analysis of the tradeoffs may be performed using L-curves or other suitable methods. This formulation allows for rapid computations and guarantees globally optimal solutions given solution existence, while promoting sparsity of the rings to inherently aid in the buildability of the magnet.

In practice, assumptions can be made to simplify the process. For example, it can be assumed that all the ring pairs have the same ring size (rₚₑᵣₘ 414), the placement of ring pairs is symmetric around z=0, each ring has the same orientation angle, and each permanent magnet element has the same orientation angle within the ring. Alternatively, this simplicity can be traded for flexibility in the design by allowing various angles, ring sizes, and asymmetric ring placement, or the like. For example, the process can be extended to accommodate multiple permanent magnet element source magnetization angles θ for each given ring or for each given magnet element.

To allow for multiple permanent magnet magnetization angles, θ, with respect to the various permanent magnet rings, the process may be extended to include a summation over multiple predefined angles, θ. The objective function may become:

$${min}_{{x}_{1} , … , {x}_{n}} {\sum }_{i} \vert {x}_{i} \vert$$

subject to:

$${B}_{z m i n} \leq {A}_{sup e r c o n} x + {\sum }_{{θ}_{k}} {C}_{perm}^{{θ}_{k}} {y}^{{θ}_{k}} \leq {B}_{z m ax}$$

$${\sum }_{{θ}_{k}} {\sum }_{j} \vert {y}_{j}^{{θ}_{k}} \vert \leq {M}_{perm} / {m}_{perm}$$

$${\sum }_{{θ}_{k}} \vert {y}_{j}^{{θ}_{k}} \vert \leq 1 ∀ j$$

For example, the process may be performed while allowing θₖ to include 0° and 90°, which would result in magnetization angle index, k, to take on values of k=[1, 2], and thus θ₁=0°, θ₂=90°. The permanent magnet mass constraint can also be modified to include a summation over all θₖ, as shown above. In practice, y^{θ}^{ₖ} can be constrained to [−1, 1] rather than [0 1], and correspondingly, θ can be constrained between [0°, 180°) rather than [0°, 360°). A value of y^{θ}^{ₖ}=−1 can be interpreted as inverting the magnetization-volume product direction of the permanent magnet ring pair (i.e., θ+180°). This can reduce the number of permanent magnet decision variables by a factor of two. The third constraint sums up the permanent magnet decision variables to prevent the process from placing multiple rings in a single candidate location 412 for all positions j.

As a non-limiting example, a tool such as linprog in MATLAB (Natick, MA) can be used to solve the design problem presented. To cast the [symbol] ₁-norm minimization problems into canonical linear programming form, auxiliary variables may be introduced to recast absolute values in the objective function. Constraints may then be rewritten as follows:

$${min}_{{t}_{1} , … , {t}_{n}} {\sum }_{i} \vert {t}_{i} \vert$$

subject to:

$${A}_{sup e r c o n} x + {\sum }_{{θ}_{k}} {C}_{perm}^{{θ}_{k}} {y}^{{θ}_{k}} \leq {B}_{z m ax} \\ - {A}_{sup e r c o n} x - {\sum }_{{θ}_{k}} {C}_{perm}^{{θ}_{k}} {y}^{{θ}_{k}} \leq - {B}_{z m i n}$$

$${\sum }_{{θ}_{k}} {\sum }_{j} {s}_{j}^{{θ}_{k}} \leq {M}_{perm} / {m}_{perm}$$

$${\sum }_{{θ}_{k}} {s}_{j}^{{θ}_{k}} \leq 1 ∀ j$$

$$\vert {x}_{i} \vert \leq {t}_{i} ∀ i$$

$$\vert {y}_{j}^{{θ}_{k}} \vert \leq {s}_{j}^{{θ}_{k}} ∀ j , k$$

where tᵢ and sⱼ^{θ}^{ₖ} are the auxiliary variables for superconducting and permanent magnet variables, respectively. Similar implementations can be applied to the superconducting only or superconducting and single permanent magnet source angles formulations.

Referring now to FIG. 5B, a block diagram illustrates the use of a design process in accordance with some aspects of the present disclosure. The design process may be performed by a computer system to design a hybrid MRI system. For example, a computer system can be used to perform an optimization to control a simulated total current in the superconducting magnet system, a simulated magnetic field profile in the target DSV, or both. The design process may also be performed physically to empirically construct a hybrid MRI system by manipulating the position of the magnet elements and superconducting windings to control the measured total current, measured magnetic field profile, or both.

A hybrid system can be defined in block 450. For example, the system can include a magnet bore that is surrounded by one or more rings of permanent magnet elements. The magnet bore may have a predetermined length and diameter. The rings may be characterized based on the radial distance from the center axis 430 of the magnet bore to the ring loop, e.g., rₚₑᵣₘ 414 as in FIG. 5A. Each ring may contain a predefined number of permanent magnet elements or a variable number of permanent magnet elements. Similarly, the orientations of each of the permanent magnet elements may be predefined (e.g., Aubert ring pair, parallel, antiparallel, or the like). Alternatively, the permanent magnet element angles, θ, may be variable based on a set of predefined angles, θₖ, as previously described.

The system may also include a plurality of superconducting windings around the magnet bore, which may form a solenoid winding, with a radial distance between the center axis of the magnet bore and the windings, e.g., rₛᵤₚ 404 as in FIG. 5A. Defining the system in block 450 may also include defining a target magnetization profile within the magnet bore or a target imaging volume (e.g., DSV 102).

A discrete spatial model of the system can be constructed by a computer system in block 452, as previously described in the context of FIG. 5A. The discrete spatial model may include discretized candidate locations for rings of permanent magnet elements (e.g., 412) and superconducting windings (e.g., 402) along the center axis, z. The spatial model may also include discretized points defining the target imaging volume (e.g., 420).

Constructing the spatial model in block 452 may also include building a physical hybrid MRI system for which, if desired, the optimization may be performed empirically. For example, rings of permanent magnet elements and superconducting windings may be placed around a magnet bore. The superconducting windings may be supplied with varying currents.

Constraints may be defined in block 454. For example, constraints may include constraining the achieved magnetic field based on a homogeneity target (e.g., ∈), constraining the total mass or weight of permanent magnet elements (e.g., Mₚₑᵣₘ), or the like. Other constraints may be used based on the desired buildability and performance of the hybrid system. For example, the bore length, bore diameter, permanent magnet element sizes or strengths, total number of permanent magnet elements, or total superconducting windings wire required may be used as constraints. Other constraints may include the permanent magnet element angles (e.g., θ) or number of permanent magnet elements in each ring.

The design process may be performed in block 456 by a computer system using a discretized spatial model or empirically using a physical system. For example, performing the design process 456 may include performing an optimization 470 by a computer system, as previously described. For computational purposes, in block 472, the optimization problem may be cast into linear programming (LP) form by introduction of auxiliary variables, as previously described. The locations of the rings and superconducting windings may be adjusted along the z axis to control the required total current in the superconducting windings, determined by experimental results or computer simulation. Additionally or alternatively, the locations along z of the rings and superconducting windings may be manipulated to control an achieved magnetic field profile.

In performing the process of block 456, an optimal hybrid system may be designed, providing superconducting winding placement 458 along the z axis, permanent magnet ring placement 460 along the z axis, permanent magnet ring element orientation angles (θ) 462, or some combination thereof. In this way, the hybrid system may achieve a highly homogeneous magnetic field of a desired field strength while reducing the required current and thereby the required superconducting windings, which may reduce system cost. Additionally or alternatively, the design process pipeline may be used to improve the magnetic field homogeneity for a given superconducting winding length.

### EXAMPLES

As a non-limiting example, the design process was performed with the following parameters:

| | Example A | |
|---|---|---|
| Superconducting Coil Parameters | | |
| z-discretization | 1.25 mm, 2.5 mm | |
| Coil diameter (ideal current loop sources) for | 1220 mm, 1180 mm | |
| superconducting magnet design | | |
| Coil diameter (ideal current loop sources) for | 1340 mm, 1300 mm | |
| hybrid magnet design | | |
| Magnet Parameters | | |
| z-discretization | 1.00 | mm |
| Cross sectional dimensions | 50 mm (x) by 62.5 mm (y) | |
| # Permanent magnet elements per ring | 48 | |
| Remanence (Bₚₑᵣₘ) range | 0 T ≤ Bₚₑᵣₘ ≤1.398 T | |
| Magnetization angle (θ) range | [0°], [0°, 180°) | |
| Other parameters | | |
| Bore lengths (in mm) | [850, 900, 950, 1000, 1050, 1100, 1150, 1200] | |
| Target field strength (B₀) | 0.5 | T |
| Target homogeneity (∈) | 5 | ppm |
| DSV Diameter | 450 | mm |

| | |
|---|---|
| # points along quarter arch DSV | 41 (~8.6 mm resolution), 111 (~3.2 mm |
| | resolution) |

### Example 1

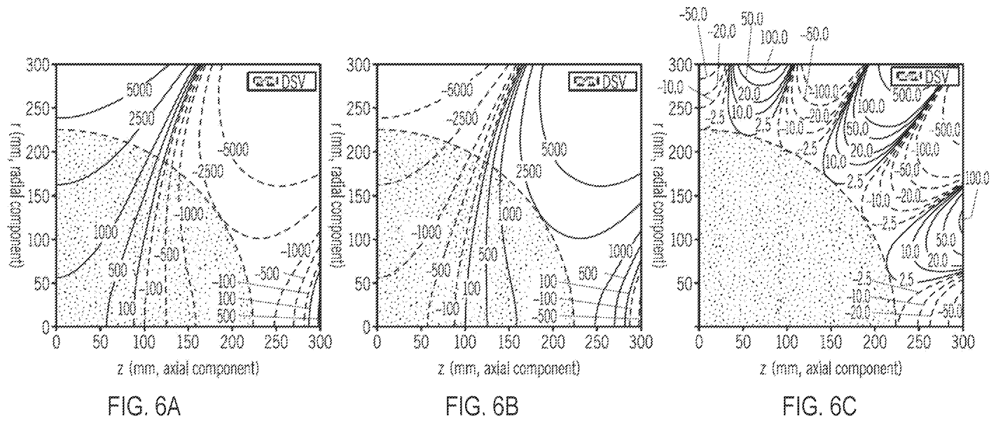

The resulting B₀ homogeneity contours for a 1000 mm bore length, 0.5 T target B₀ field at 5 ppm target homogeneity, and maximum of 250 kg of magnet elements are shown in FIGS. 6A, 6B, and 6C for Example A. FIG. 6A shows the homogeneity contours from a superconducting magnet system. FIG. 6B shows the homogeneity contours from the Aubert rings only. FIG. 6C shows the homogeneity contours for hybrid system combining the superconducting magnet system with the Aubert rings. As shown, the superconducting magnet design is highly inhomogeneous at the wire length chosen (2.94*10⁶ ampere turns). However, when the 250 kg of Aubert rings are added, the system yields the desired 5 ppm homogeneity. Because the Aubert rings improve homogeneity, the total current required for the hybrid magnet design is reduced.

### Example 2

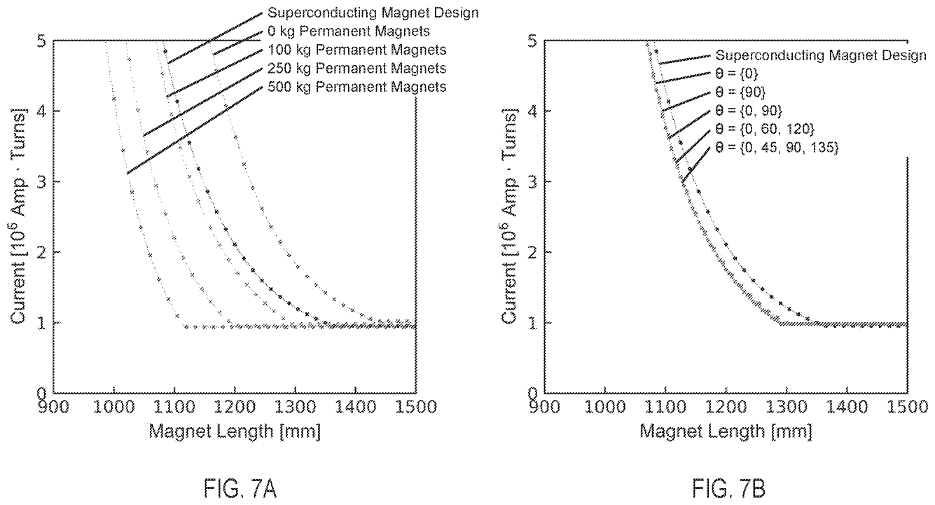

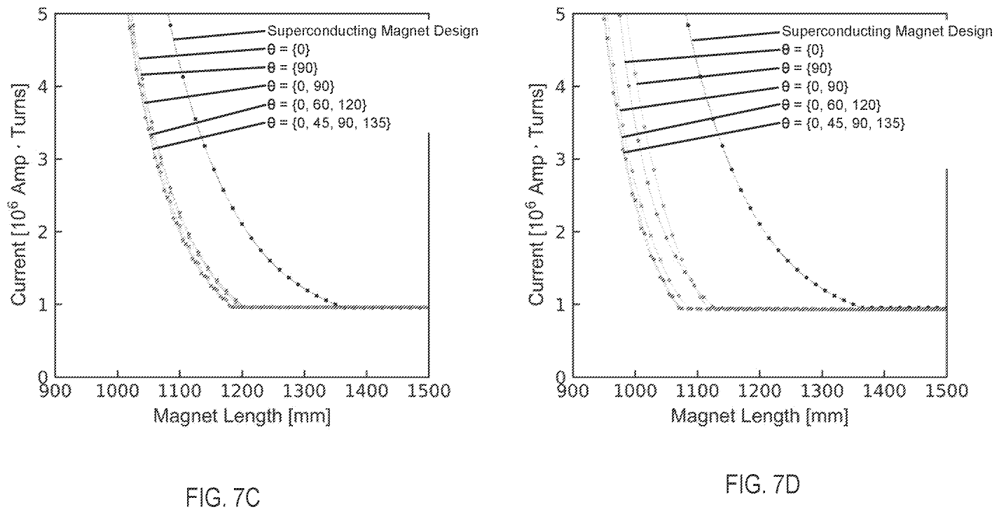

FIG. 7A shows L-curves that illustrate the tradeoffs between constrained length and maximum magnet mass using Aubert rings in the permanent magnet space. Increasing the maximum magnet mass reduces the required number of superconducting magnet windings or the required bore length to achieve at least 5 ppm homogeneity at 0.5 T.

FIG. 7B shows L-curves that illustrate the tradeoffs between constrained length and various angle configurations for no more than 100 kg of allowed permanent magnet mass. The hybrid magnet designs with all four configurations achieve the target 0.5 T B₀ field with a maximum 5 ppm inhomogeneity with less superconducting wire, thereby reducing costs, or reduced bore length, which increases exam flexibility and reduces claustrophobia. For example, operating at the optimal magnet length (plateau section of L-curves), 100 kg of permanent magnet mass results in ˜5.13% reduction of optimal magnet length over all angle configurations.

FIG. 7C shows L-curves that illustrate the tradeoffs between constrained length and various angle configurations for no more than 250 kg of allowed permanent magnet mass for a 5 ppm homogeneity specification at 0.5 T. Increasing the allowed mass over 100 kg in FIG. 7B results in an additional reduction in optimal magnet length, for up to 12.1% reduction in the 250 kg allowed mass case.

FIG. 7D shows L-curves that illustrate the tradeoffs between constrained length and various angle configurations for no more than 500 kg of allowed permanent magnet mass for a 5 ppm homogeneity specification at 0.5 T. Increasing the allowed mass over 100 kg and 250 kg in FIG. 7B, C again results in further reduction of optimal magnet length. Furthermore, enhanced reduction for more complex angle configurations can be seen in this higher allowed permanent magnet mass case. For the single angle configurations of {0}, {90}, approximately 18% reduction in optimal magnet length is observed. For multiple angle configurations such as {0, 45, 90, 135}, up to 21.2% reduction in optimal magnet length can be observed.

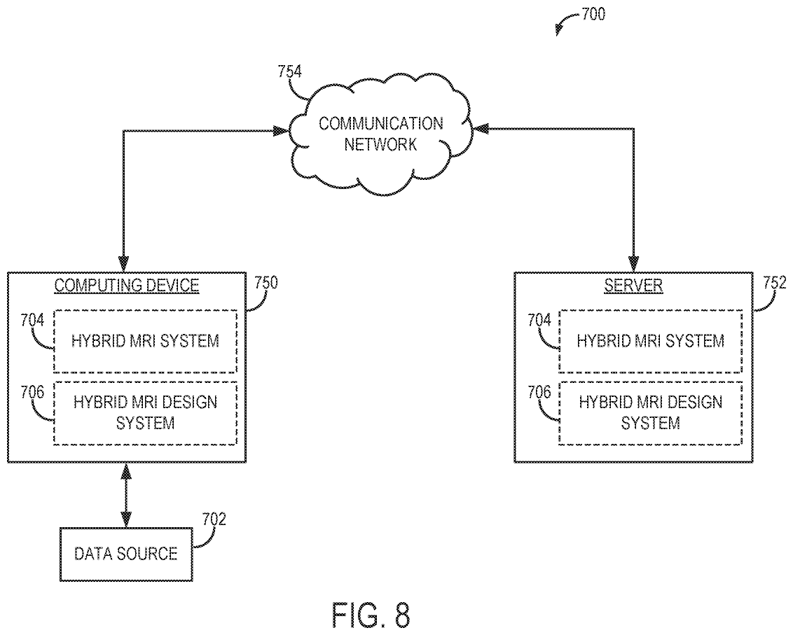

Referring now to FIG. 8, an example of a system 700 for generating MRI images and designing a hybrid MRI system in accordance with some embodiments of the systems and methods described in the present disclosure is shown. As shown in FIG. 8, a computing device 750 can receive one or more types of data (e.g., k-space data, training data) from data source 702, which may be a k-space data source. In some embodiments, computing device 750 can execute at least a portion of a hybrid MRI system 704 to reconstruct magnetic resonance images from k-space data received from the data source 702. In some embodiments, computing device 750 can execute at least a portion of a hybrid MRI design system 706 to design a hybrid MRI system.

Additionally or alternatively, in some embodiments, the computing device 750 can communicate information about data received from the image data source 702 to a server 752 over a communication network 754, which can execute at least a portion of the hybrid MRI system 704 or the hybrid MRI design system 706. In such embodiments, the server 752 can return information to the computing device 750 (and/or any other suitable computing device) indicative of an output of the MRI reconstruction or the design.

In some embodiments, computing device 750 and/or server 752 can be any suitable computing device or combination of devices, such as a desktop computer, a laptop computer, a smartphone, a tablet computer, a wearable computer, a server computer, a virtual machine being executed by a physical computing device, and so on. The computing device 750 and/or server 752 can also reconstruct images from the data.

In some embodiments, data source 702 can be any suitable source of data (e.g., measurement data, images reconstructed from measurement data), such as an MRI system, another computing device (e.g., a server storing k-space data), and so on. In some embodiments, data source 702 can be local to computing device 750. For example, data source 702 can be incorporated with computing device 750 (e.g., computing device 750 can be configured as part of a device for measuring, recording, estimating, acquiring, or otherwise collecting or storing data). As another example, data source 702 can be connected to computing device 750 by a cable, a direct wireless link, and so on. Additionally or alternatively, in some embodiments, data source 702 can be located locally and/or remotely from computing device 750, and can communicate data to computing device 750 (and/or server 752) via a communication network (e.g., communication network 754).

In some embodiments, communication network 754 can be any suitable communication network or combination of communication networks. For example, communication network 754 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 754 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, and so on.

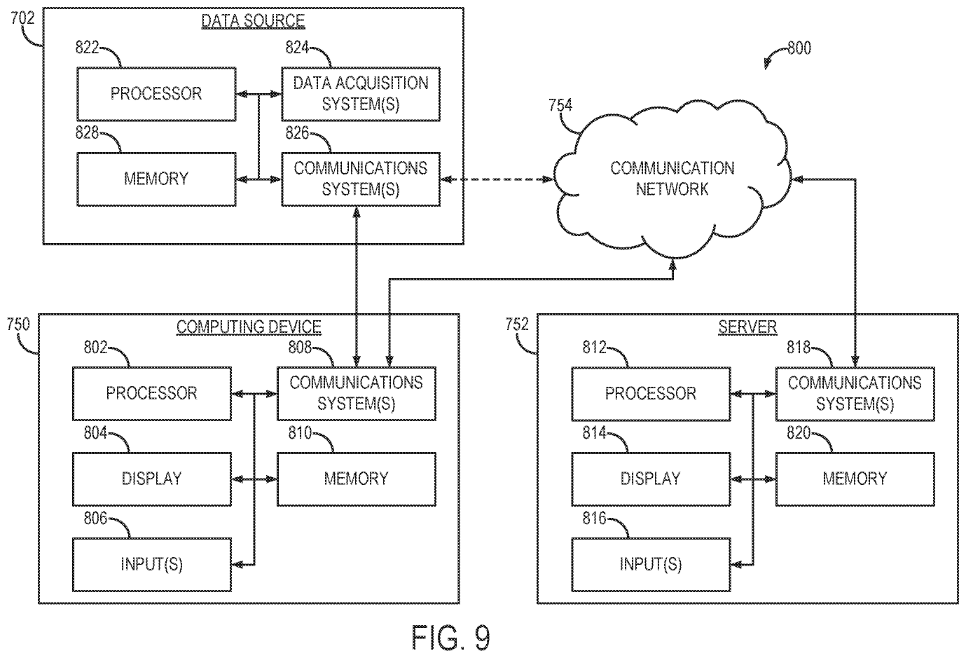

Referring now to FIG. 9, an example of hardware 800 that can be used to implement data source 702, computing device 750, and server 752 in accordance with some embodiments of the systems and methods described in the present disclosure is shown.

As shown in FIG. 9, in some embodiments, computing device 750 can include a processor 802, a display 804, one or more inputs 806, one or more communication systems 808, and/or memory 810. In some embodiments, processor 802 can be any suitable hardware processor or combination of processors, such as a central processing unit (“CPU”), a graphics processing unit (“GPU”), and so on. In some embodiments, display 804 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 806 can include any suitable input devices and/or sensors that can be used to receive user input, such as a keyboard, a mouse, a touchscreen, a microphone, and so on.

In some embodiments, communications systems 808 can include any suitable hardware, firmware, and/or software for communicating information over communication network 754 and/or any other suitable communication networks. For example, communications systems 808 can include one or more transceivers, one or more communication chips and/or chip sets, and so on. In a more particular example, communications systems 808 can include hardware, firmware, and/or software that can be used to establish a Wi-Fi connection, a Bluetooth connection, a cellular connection, an Ethernet connection, and so on.

In some embodiments, memory 810 can include any suitable storage device or devices that can be used to store instructions, values, data, or the like, that can be used, for example, by processor 802 to present content using display 804, to communicate with server 752 via communications system(s) 808, and so on. Memory 810 can include any suitable volatile memory, non-volatile memory, storage, or any suitable combination thereof. For example, memory 810 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 810 can have encoded thereon, or otherwise stored therein, a computer program for controlling operation of computing device 750. In such embodiments, processor 802 can execute at least a portion of the computer program to present content (e.g., images, user interfaces, graphics, tables), receive content from server 752, transmit information to server 752, and so on. For example, the processor 802 and the memory 810 can be configured to perform the methods described herein (e.g., the design method illustrated in FIGS. 5A and 5B).

In some embodiments, server 752 can include a processor 812, a display 814, one or more inputs 816, one or more communications systems 818, and/or memory 820. In some embodiments, processor 812 can be any suitable hardware processor or combination of processors, such as a CPU, a GPU, and so on. In some embodiments, display 814 can include any suitable display devices, such as an LCD screen, LED display, OLED display, electrophoretic display, a computer monitor, a touchscreen, a television, and so on. In some embodiments, inputs 816 can include any suitable input devices and/or sensors that can be used to receive user input, such as a keyboard, a mouse, a touchscreen, a microphone, and so on.

In some embodiments, communications systems 818 can include any suitable hardware, firmware, and/or software for communicating information over communication network 754 and/or any other suitable communication networks. For example, communications systems 818 can include one or more transceivers, one or more communication chips and/or chip sets, and so on. In a more particular example, communications systems 818 can include hardware, firmware, and/or software that can be used to establish a Wi-Fi connection, a Bluetooth connection, a cellular connection, an Ethernet connection, and so on.

In some embodiments, memory 820 can include any suitable storage device or devices that can be used to store instructions, values, data, or the like, that can be used, for example, by processor 812 to present content using display 814, to communicate with one or more computing devices 750, and so on. Memory 820 can include any suitable volatile memory, non-volatile memory, storage, or any suitable combination thereof. For example, memory 820 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 820 can have encoded thereon a server program for controlling operation of server 752. In such embodiments, processor 812 can execute at least a portion of the server program to transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone), and so on.

In some embodiments, the server 752 is configured to perform the methods described in the present disclosure. For example, the processor 812 and memory 820 can be configured to perform the methods described herein (e.g., the design method illustrated in FIGS. 5A and 5B).

In some embodiments, data source 702 can include a processor 822, one or more data acquisition systems 824, one or more communications systems 826, and/or memory 828. In some embodiments, processor 822 can be any suitable hardware processor or combination of processors, such as a CPU, a GPU, and so on. In some embodiments, the one or more data acquisition systems 824 are generally configured to acquire data, images, or both, and can include an MRI system. Additionally or alternatively, in some embodiments, the one or more data acquisition systems 824 can include any suitable hardware, firmware, and/or software for coupling to and/or controlling operations of an MRI system. In some embodiments, one or more portions of the data acquisition system(s) 824 can be removable and/or replaceable.

Note that, although not shown, data source 702 can include any suitable inputs and/or outputs. For example, data source 702 can include input devices and/or sensors that can be used to receive user input, such as a keyboard, a mouse, a touchscreen, a microphone, a trackpad, a trackball, and so on. As another example, data source 702 can include any suitable display devices, such as an LCD screen, an LED display, an OLED display, an electrophoretic display, a computer monitor, a touchscreen, a television, etc., one or more speakers, and so on.

In some embodiments, communications systems 826 can include any suitable hardware, firmware, and/or software for communicating information to computing device 750 (and, in some embodiments, over communication network 754 and/or any other suitable communication networks). For example, communications systems 826 can include one or more transceivers, one or more communication chips and/or chip sets, and so on. In a more particular example, communications systems 826 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 828 can include any suitable storage device or devices that can be used to store instructions, values, data, or the like, that can be used, for example, by processor 822 to control the one or more data acquisition systems 824, and/or receive data from the one or more data acquisition systems 824; to generate images from data; present content (e.g., images, a user interface) using a display; communicate with one or more computing devices 750; and so on. Memory 828 can include any suitable volatile memory, non-volatile memory, storage, or any suitable combination thereof. For example, memory 828 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 828 can have encoded thereon, or otherwise stored therein, a program for controlling operation of data source 702. In such embodiments, processor 822 can execute at least a portion of the program to generate images, transmit information and/or content (e.g., data, images) to one or more computing devices 750, receive information and/or content from one or more computing devices 750, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A system for magnetic resonance imaging (MRI), the system comprising:
a bore configured to receive a subject for imaging during an MRI process;
superconducting wires configured to produce a main magnetic field within the bore;
a cryostat configured to maintain the superconducting wires at a temperature that maintains the superconducting wires in a superconducting operational state during the MRI process;
a plurality of permanent magnet elements configured to supplement or shape the main magnetic field within the bore;
a gradient coil set configured to apply a magnetic gradient onto the main magnetic field; and
a radiofrequency system configured to transmit and receive radiofrequency signals during the MRI process.

2. The system of claim 1, wherein the permanent magnet elements are arranged in one or more ring pairs positioned proximate to the bore such that each ring of the ring pairs surrounds at least a part of a circumference of the bore.

3. A system for magnetic resonance imaging (MRI), the system comprising:
a bore configured to receive a subject for imaging during an MRI process;
superconducting wires arranged in a solenoid and configured to produce a main magnetic field within the bore;
a cryostat configured to maintain the superconducting wires at a temperature that maintains the superconducting wires in a superconducting operational state during the MRI process; and
a plurality of permanent magnet elements configured to supplement or shape the main magnetic field within the bore.

4. The system of claim 3, wherein:
the permanent magnet elements are arranged outside of the bore;
the cryostat is arranged outside the permanent magnet elements; and
the superconducting wires are arranged outside the cryostat.

5. The system of claim 4, further comprising a gradient coil set configured to apply a magnetic gradient onto the main magnetic field.

6. The system of claim 3, wherein the permanent magnet elements include neodymium.

7. The system of claim 3, wherein the permanent magnet elements form at least one pair of rings, and wherein the rings have a circumference that extends around the bore.

8. The system of claim 3, wherein the permanent magnet elements surround at least a portion of the bore.

9. The system of claim 3, wherein the permanent magnet elements are arranged in a pair of rings in an Aubert ring configuration.

10. The system of claim 3, wherein the permanent magnet elements are characterized by magnetic moments oriented parallel to an axis parallel to a longitudinal axis of the bore.

11. The system of claim 3, wherein the permanent magnet elements are characterized by magnetic moments oriented antiparallel to an axis parallel to a longitudinal axis of the bore.

12. A method for manufacturing a hybrid magnetic resonance imaging (MRI) system, the method comprising:
providing a bore;
arranging a superconducting magnet system proximate to the bore to form a static magnetic field within the bore; and
arranging one or more rings of permanent magnet elements proximate to the bore to supplement or shape the static magnetic field.

13. The method of claim 12, further comprising:
creating a discrete spatial model of the MRI system, which comprises a first distance defining a radial distance between a center axis of the bore and the permanent magnet elements and a second distance defining a radial distance between a center axis of the bore and the superconducting magnet system; and
controlling at least one of a simulated total current in the superconducting magnet system or a simulated magnetic field profile in a target imaging volume by manipulating a position along the center axis of at least one of the permanent magnet elements or the superconducting magnet system.

14. The method of claim 13, further comprising defining a constraint comprising at least one of a total permanent magnet weight or a homogeneity target; and
wherein controlling at least one of a simulated total current in the superconducting magnet system or a simulated magnetic field profile in a target imaging volume is constrained using the discrete spatial model and a discretized target imaging volume.

15. The method of claim 14, wherein controlling at least one of a simulated total current in the superconducting windings or a simulated magnetic field profile comprises defining an optimization problem with an objective function defined as
$${min}_{{x}_{1} , … , {x}_{n}} {\sum }_{i} \vert {x}_{i} \vert ,$$
wherein xᵢ is and i^{th} superconducting coil current of the superconducting magnet system and n is a number of discretized candidate locations for superconducting coil current;
wherein the constraint of the homogeneity target is defined as:
$${B}_{0} ( 1 - 0.5 ϵ ) \leq {A}_{sup} x + {C}_{perm} y \leq {B}_{0} ( 1 + 0.5 ϵ )$$
wherein B₀ is a target field strength, ∈ is a homogeneity target, Aₛᵤₚ is a simulated target field per unit current, x is a simulated coil current, Cₚₑᵣₘ is a target field per unit remanence, and y is a remanence of one or more rings formed by the permanent magnet elements; and
wherein the constraint of the total permanent magnet weight is defined as:
$${\sum }_{j} {m}_{p e r m} \vert {y}_{j} \vert \leq {M}_{p e r m}$$
wherein yⱼ is a j^{th} remanence, mₚₑᵣₘ is a discretized unit mass of the rings formed by the permanent magnet elements, and Mₚₑᵣₘ is a maximum mass of the combined one or more rings formed by the permanent magnet elements.

16. The method of claim 15 wherein the optimization problem comprises casting the superconducting coil current and remanences into linear programming by introducing auxiliary variables, wherein casting absolute values comprises casting into two linear inequalities.

17. The method of claim 13, wherein controlling at least one of a simulated total current in the superconducting magnet system or a simulated magnetic field profile further comprises manipulating an orientation angle of at least one of the permanent magnet elements with respect to the center axis.

18. The method of claim 13, wherein the second radial distance is larger than the first radial distance.

19. A method for manufacturing a hybrid magnetic resonance imaging (MRI) system comprising:
constructing a bore with a center axis;
arranging one or more rings of magnet elements around the bore at a first radial distance between the center axis and the one or more rings;
arranging a plurality of superconducting windings around the bore at a second radial distance between the center axis and the windings; and
manipulating a position along the center axis of at least one of the one or more rings of magnet elements or the plurality of superconducting windings to control at least one of a required total current in the plurality of superconducting windings or an achieved magnetic field profile within a target imaging volume.

20. The method of claim 19, wherein the magnet elements are permanent magnet elements.

21. The method of claim 20, further comprising defining a constraint comprising at least one of a total permanent magnet weight or a homogeneity target, and wherein controlling at least one of a simulated total current in the superconducting winding or a simulated magnetic field profile in a target imaging volume is constrained by the constraint.

22. The method of claim 20, wherein the second radial distance is larger than the first radial distance.

23. The method of claim 19, wherein the magnet elements are non-superconducting magnets.
